(19) United States
(12) Patent Application Publication
(10) Pub. No.: US 2019/0219556 A1
(43) Pub. Date: Jul. 18, 2019

(54) METHOD AND SYSTEM FOR MEASURING THE ENERGY CONTENT OF GAS

(71) Applicant: SP TECHNICAL RESEARCH INSTITUTE OF SWEDEN, Borås (SE)

(72) Inventor: Oliver BUKER, Borås (SE)

(73) Assignee: SP TECHNICAL RESEARCH INSTITUTE OF SWEDENå, Borås (SE)

(21) Appl. No.: 16/335,080

(22) PCT Filed: Sep. 19, 2017
(86) PCT No.: PCT/EP2017/073642

§ 371 Date: Mar. 20, 2019

(30) Foreign Application Priority Data
Sep. 20, 2016 (EP) 16189761.6

(51) Int. Cl. G01N 33/22; G01N 29/024; G01N 29/22; G01F 23/284; G01N 22/00; G01S 13/88

(52) U.S. Cl. CPC G01N 33/225; G01N 29/024; G01N 29/22; G01N 2291/02836; G01N 22/00; G01S 13/88; G01F 23/284

(57) ABSTRACT

A method of measuring the energy content of a gas comprises measuring the speed of sound in the gas; measuring the relative permittivity of the gas using a guided wave radar or non-guided wave (non-contacting) radar sensor; and correlating the measured speed of sound and relative permittivity to derive the energy content of the gas. A system for measuring the energy content of a gas comprises a first sensor for measuring the speed of sound in the gas; a second sensor for measuring the relative permittivity of the gas using a guided wave radar or non-guided wave (non-contacting) radar sensor; and a correlator configured to correlate the measured speed of sound and relative permittivity to derive the energy content of the gas. The carbon dioxide content of the gas may also be measured with a suitable sensor and used in the correlation.

### FIELD OF THE INVENTION

This invention relates to methods and systems for measuring the energy content of a fluid such as a hydrocarbon fuel gas flowing in a pipeline.

### BACKGROUND

The energy content of fuel gases such as natural gas (NG), liquefied natural gas (LNG), compressed natural gas (CNG), biogas, etc. can vary as a result of variations in the composition of the gases, both in terms of hydrocarbon mix, and in terms of the content of calorifically-inert gases such as nitrogen and carbon dioxide. Customers for fuel gases pay according to the energy of the delivered gas. This means it is necessary to determine the mass or volume and the energy content of the gas (expressed as energy per unit mass or unit volume) delivered so that the total energy delivery can be determined. Consequently, a simple (mass or volumetric) flow measurement will only provide the delivered mass or volume and not an accurate value for the energy content of the gas delivered through a pipeline. Such measurement is sometimes known as “fiscal” monitoring or measurement, for example fiscal metering used for custody transfer.

The energy content or calorific value (the term “energy content” used herein includes calorific value and heat value) of gases can be determined calorimetrically, but it has been difficult to provide such measurements on-line for flowing gases. Alternatively, the chemical composition of the gases can be determined by gas chromatography (GC). This allows the energy content to be calculated. However, again such systems have been difficult to provide as an on-line, (quasi) real time measurement and the instruments are complex and expensive.

In order to avoid the problems of full composition analysis, or direct calorimetry, a number of techniques known as “correlation” measurement have been proposed. In correlation measurements, a series of “incomplete” measurement values are obtained, such as physical properties or measurements of specific components, and correlations developed that indicate the energy content of the gas providing such values. The accuracy of such techniques depends on the parameters selected for measurement and the techniques used to measure the parameters. While correlation measurements can provide reasonable accuracy, the complexity of some of the measurement technologies, and their costs have meant that adoption has been limited to date.

This invention aim to provide a correlation measurement technique that is based on robust and widely available technologies with the aim of reducing lifecycle costs.

### SUMMARY

A first aspect of the invention provides a method of measuring the energy content of a gas, comprising: measuring the speed of sound in the gas; estimating the relative permittivity (dielectric constant)—approximately measured in terms of the group refractive index—of the gas using a guided wave radar sensor; and correlating the measured speed of sound and relative permittivity to derive the energy content of the gas.

Where the gas is flowing in a conduit, the measurements of speed of sound and relative permittivity are made on the flowing gas. The measurement can further comprise determining volume, pressure, and temperature parameters of the flowing gas, and using the parameters to derive the energy content of the gas.

Estimating the relative permittivity of the gas—approximately measured in terms of the group refractive index—can comprise a time domain reflectometry measurement of a microwave pulse in a wave guide.

Guided wave radar sensors are widely available as level sensors for process monitoring, typically being used to measure the level of a liquid or solid in a container or process line. The time of flight measurement in a guided wave radar sensor is dependent on the relative permittivity of the medium though which the wave propagates.

The method can also comprise measuring the carbon dioxide content of the gas, and correlating the measured speed of sound, relative permittivity, and carbon dioxide content to derive the energy content of the gas.

The gas can be natural gas (NG), liquefied natural gas (LNG), compressed natural gas (CNG) or biogas. Where the gas is LNG, it may not be necessary to measure carbon dioxide content. The term “gas” used herein includes not only hydrocarbons in the gas phase, such as might be used for gas fuel, but also the liquefied forms, such as LNG, typically used for storage and transportation.

A second aspect of the invention provides a system for measuring the energy content of a gas, comprising a first sensor for measuring the speed of sound in the gas; a second sensor for measuring the relative permittivity of the gas using a guided wave radar sensor; and a correlator configured to correlate the measured speed of sound and relative permittivity to derive the energy content of the gas.

The system can further comprise a conduit housing the sensors and through which the gas flows. The system can also comprise further sensors for determining volume, pressure, and temperature parameters of the flowing gas, wherein the correlator is configured to use the parameters to derive the energy content of the gas.

The first sensor can comprise an ultrasonic flow meter or another sensor which is capable to measure the speed of sound

The second sensor can comprise a microwave source and a waveguide of predetermined configuration, the second sensor being configured to provide a time domain reflectometry measurement of a microwave pulse reflected by a predetermined feature of the waveguide configuration, such as the end of a reference rod mounted on the waveguide, for determining the relative permittivity of the gas—approximately estimated in terms of the group refractive index.

The system can also comprise a third sensor, such as an infrared sensor, for measuring the carbon dioxide content of the gas, and wherein the correlator is configured to use the measured speed of sound, relative permittivity (dielectric constant) and carbon dioxide content to derive the energy content of the gas.

The system can also comprise a processor configured to receive the output of the correlator as an input, and to output values of superior calorific value, Wobbe index and normal density of the gas.

The speed of sound measurement may be replaced by a density measurement in certain cases. The system may also comprise additional sensors to provide multiple measurements of a given parameter, or measurements of further parameters depending on requirements.

Other aspects of the invention will be apparent from the following description.

### DRAWINGS

FIG. 1 shows a schematic view of a guided wave radar level measurement system;

FIG. 2 shows a plot of levels in methane obtained with a system corresponding to FIG. 1;

FIG. 3 shows a plot of measured and theoretical dielectric constant with respect to pressure for nitrogen and methane;

FIG. 4 shows a schematic view of a system for determining the energy content of a gas.

### DESCRIPTION

Various techniques for correlation measurements to determine the energy content of gases have been proposed previously. Examples can be found in “Thermodynamic research improves energy measurement of natural gas” M. Jaeschke, Thermochimica Acta 382 (2002) 37-45, and in U.S. Pat. No. 627,380 and US 2002/0124630. Methods of determining gas density from refractive index and dielectric constant measurements have been developed and reviewed for instance in “Refractometry and gas density” L R Pendrill, Metrologia—Special Issue “Density Metrology” (2004), 41(2): S40-S51. Among the techniques disclosed, measurements of relative permittivity or dielectric constant (εᵣ) are disclosed. A re-entrant cavity device operated as an LC resonator is disclosed for deriving relative permittivity values. Such devices have high production costs and technical tolerances and so have not been widely adopted for such use.

This invention is based on the recognition that measurements made using guided wave radar (GWR) and non-GWR (non-contacting radar) devices can provide the necessary information for the relative permittivity of a gas phase to be determined with sufficient accuracy for energy content monitoring.

In recent years GWR and non-GWR (non-contacting radar) devices have become a popular method of measuring levels such as liquid levels in process equipment, for example within the oil and gas industry. GWR level sensors are considered to offer reliability and maintenance advantages over many other measurement technologies that have traditionally been used. In general GWR level sensors are seen as being unaffected by the properties of the gas present above the liquid at many process conditions. This is in fact only partially true and elevated gas densities can lead to a loss of accuracy when using GWR or non-GWR (non-contacting radar) for level measurement.

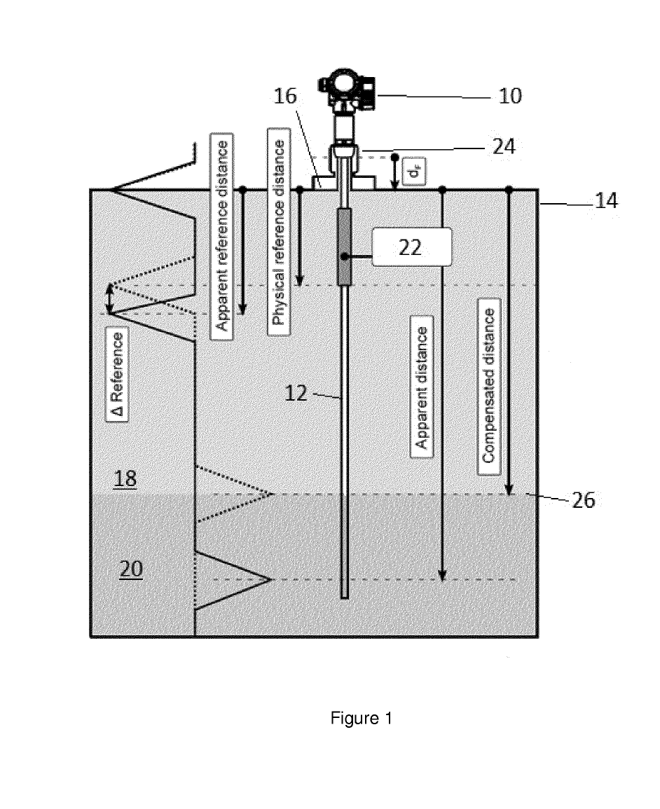

GWR and non-GWR (non-contacting radar) level sensors are time-domain reflectometer (TDR) devices (time-of-flight measuring device). An example of such a device is show in FIG. 1 and comprises an electronics module 10 including a microwave signal source and a waveguide 12. The device is mounted in process equipment 14, such as a tank or pipeline, such that the electronics module 10 is mounted on an outer surface of the process equipment 14 at a mounting flange 16. The waveguide 12 projects inside the process equipment 14 such that it extends through a gas phase 18 and into a liquid phase 20. In the waveguide configuration shown in FIG. 1, a reference feature comprising a reference rod 22 is provided inside the process equipment below the mounting flange 16. A short section 24 of the waveguide 12 extends through the flange outside the process equipment 14, the electronics module 10 being mounted on the end of this short section 24. In normal use, the waveguide is positioned such that its inner end is located below the surface 26 of the liquid phase 20.

In use, a microwave pulse is emitted from the electronics module 10 and travels down the waveguide 12 to the surface of the liquid 26. At the surface 26, a portion of the energy is reflected and this travels back along the waveguide 12 to the electronics module 10 where it is detected. If the velocity ν of the microwave pulse is known, and the time t between emitting the pulse and the receiving the reflection is measured, then the distance d to the liquid surface can be calculated using equation 1. From this measured distance the liquid level is then calculated and output by the transmitter.

$$d = \frac{v \cdot t}{2} \qquad ( 1 )$$

In a vacuum an electromagnetic wave will travel at the speed of light c which is related to the vacuum permittivity ε₀ and permeability μ₀ by the relationship shown in equation 2.

$$c = \frac{1}{\sqrt{{ɛ}_{0} {μ}_{0}}} \qquad ( 2 )$$

An electromagnetic field will be affected by the gas phase compared with in vacuo.
a.) A static (DC) electric field will be modified by the relative permittivity εᵣ of the gas. The dielectric constant of a gas, as measured for instance by Harvey A H, Lemmon E W, “Method for Estimating the Dielectric Constant of Natural Gas Mixtures”, International Journal of Thermophysics 2005; 26(1): 31-46, can be related to the phase velocity νₚₕ(0) since a DC field can be assumed as electromagnetic wave with infinite wavelength at ω=0.
   In this case equation 3a relates the phase velocity to the material properties by the use of the relative permittivity εᵣ(0) and relative permeability μᵣ(0).

$${v}_{ph} ( 0 ) = \frac{1}{\sqrt{{ɛ}_{0} {ɛ}_{r} ( 0 ) {μ}_{0} {μ}_{r} ( 0 )}} \qquad ( 3 a )$$

b.) The phase velocity of an oscillating and continuous electromagnetic field (AC) will be modified by the (phase) refractive index nₚₕ(ω) of the gas, such that the velocity will no longer be the speed of light but will have a lower velocity νₚₕ(0).
   In this case equation 3b relates the velocity to the material properties by the use of the relative permittivity εᵣ(ω) and relative permeability μᵣ(ω).

$${v}_{ph} ( ω ) = \frac{1}{\sqrt{{ɛ}_{0} {ɛ}_{r} ( ω ) {μ}_{0} {μ}_{r} ( ω )}} = \frac{c}{\sqrt{{ɛ}_{r} ( ω ) {μ}_{r} ( ω )}} = \frac{c}{{n}_{ph} ( ω )} \qquad ( 3 b )$$

c.) The group velocity ν_{gr}(ω) of an oscillating electromagnetic field (AC) will be modified by the (group) refractive index n_{gr}(ω) of the gas:

$${n}_{gr} ( ω ) = \frac{c}{{v}_{gr} ( ω )} .$$

For GWR and non-GWR (non-contacting radar) sensors the group velocity is the velocity with which the envelope of the pulse (better known as modulation) propagates in the media. It is well-known that the group and phase refractive indices are related to each other, where for a medium of dispersion

$$\frac{\partial {n}_{ph} ( ω )}{\partial λ}$$

follows:

$${n}_{gr} ( ω ) = \frac{{n}_{ph} ( ω )}{1 + \frac{λ}{{n}_{ph} ( ω )} \cdot \frac{\partial {n}_{ph} ( ω )}{\partial λ}} \approx {n}_{ph} ( ω ) - λ \cdot \frac{\partial {n}_{ph} ( ω )}{\partial λ} \qquad ( 3 c )$$

For most transparent materials μᵣ(ω)=μᵣ(0)≈1. Taking this into account and combining equations 2 and 3b it can be stated that the velocity varies with the square root of the relative permittivity.

$${v}_{ph} ( ω ) = \frac{c}{\sqrt{{ɛ}_{r} ( ω )}} \qquad ( 4 )$$

The phase and group velocities depend on the working frequency ω of the GWR or non-GWR (non-contacting radar) sensor. The challenge is to convert the measured (AC) frequency-dependent relative permittivity values εᵣ(ω) from the estimated phase and measured group indices into the values for εᵣ(0), to be combined with the known values published for instance, by Harvey A H, Lemmon E W, “Method for Estimating the Dielectric Constant of Natural Gas Mixtures”, International Journal of Thermophysics 2005; 26(1): 31-46.

If the process always contains the same gas type and runs at a constant gas density then it is relatively easy to take account of the reduced propagation speed within the control system, but there are inherent problems with using this method:
The delay caused by the gas phase must be known.
It may be possible to calculate the level correction required using published gas data but this is complex for mixtures of gases.
During operation changes in the gas mixture, the temperature or the pressure can lead to considerable errors.

One way to consistently compensate for this effect is to directly measure the propagation speed through the gas and continuously compensate for it.

The system of FIG. 1 has integrated automatic gas phase compensation. This integrated function provides the possibility to correct the level information regarding temperature and pressure changes, which in turn can be related to the change of the relative permittivity of the gas above the liquid surface.

The principle adopted for automatic gas phase compensation is to present a target, the reference rod 22 positioned at a fixed distance along the waveguide as shown in FIG. 1.

The step decrease in diameter of the waveguide 12 due to the reference rod 22 creates a radar echo at a known physical distance, the “Physical reference distance”. It can be seen that as the gas pressure is increased, and hence the gas relative dielectric, the radar echoes received from the reference target 22 “Apparent reference distance” and the liquid surface 26 “Apparent distance” are shifted downwards appearing to be at a greater distance. Both echoes are shifted by the same factor, therefore the further the distance the greater the shift seen as is evident from the greater shift seen in the measured distance compared to the reference distance. As the delay through the gas phase is measured directly by the reference section this will correct the measured distance “Compensated distance” regardless of the properties of the gas phase across the full temperature and pressure range. This measurement allows the introduction of a so-called “microfactor” that is related to the speed with which the electromagnetic signal propagates along the waveguide 12. A microfactor of 1 means that the signal propagates with the speed of light. In the case of the system of FIG. 1, the gas phase compensation is calculated from the apparent shift ΔReference in the measured distance to the echo from the end of the reference section 22 caused by the slower wave propagation speed:

$$microfactor = \frac{Physical reference distance}{Apparent reference distance} \qquad ( 5 )$$

This automatic gas phase compensation method can also be used to measure the density-dependent relative permittivity (=dielectric constant) εᵣ of the gas phase 18 since the microfactor is directly related to the relative permittivity:

$${ɛ}_{r} = \frac{1}{{microfactor}^{2}} \qquad ( 6 )$$

The determination of the relative permittivity εᵣ takes place automatically through the determination of the microfactor value (see equation 5) and the conversion of the microfactor value to the relative permittivity by using equation 6.

The Clausius-Mossotti equation relates the macroscopic property relative dielectric constant εᵣ to the microscopic property polarisability α:

$${P}_{m} = \frac{{ɛ}_{r} - 1}{{ɛ}_{r} + 2} \cdot \frac{1}{{ρ}_{m}} = \frac{{N}_{A} α}{3 {ɛ}_{0}} = \frac{4 π}{3} {N}_{A} \cdot {α}^{′} \qquad ( 7 )$$

The molar polarisation Pₘ is nearly independent from the molar density ρₘ. Many gases show a small but significant deviation from an ideal gas and, therefore, from equation 7. These deviations can be considered by means of virial expansion. The virial coefficients characterise interactions between the particles in the system, which makes it possible to represent the temperature and density dependency of the molar polarisation:

$${P}_{m} = \frac{{ɛ}_{r} - 1}{{ɛ}_{r} + 2} \cdot \frac{1}{{ρ}_{m}} = {A}_{ɛ} + {B}_{ɛ} \cdot {ρ}_{m} + {C}_{ɛ} \cdot {ρ}_{m}^{2} + … \qquad ( 8 )$$

A_{ε}=4π·N_{A}α′/3=N_{A}α/3ε₀ is the first virial coefficient and B, and C, are the second and third virial coefficients. Equation 8 is valid for non-polar gases. For molecules with a permanent dipole moment u, an additional term contributes to the low-density expansion of the molar polarisation according to the Debye equation:

$${P}_{m} = \frac{{ɛ}_{r} - 1}{{ɛ}_{r} + 2} \cdot \frac{1}{{ρ}_{m}} = \frac{{N}_{A}}{3 {ɛ}_{0}} ( α + \frac{{μ}^{2}}{3 {k}_{B} T} ) = {A}_{ɛ} + {A}_{p} \qquad ( 9 )$$

A_{ε}=N_{A}μ²/3ε₀k_{B}T is due to the contribution from the permanent dipole moment, which is negligible when the medium is non-polar. Equation 9 can also be expressed by means of virial coefficients, which results in:

$${P}_{m} = \frac{{ɛ}_{r} - 1}{{ɛ}_{r} + 2} \cdot \frac{1}{{ρ}_{m}} = {A}_{ɛ} + {A}_{μ} + {B}_{ɛ} \cdot {ρ}_{m} + {C}_{ɛ} \cdot {ρ}_{m}^{2} + … \qquad ( 10 )$$

Because higher-order terms are difficult to extract from data, an empirical form was chosen to extend the correlation to high densities. The final form of the correlation given by Harvey A H, Lemmon E W, “Method for Estimating the Dielectric Constant of Natural Gas Mixtures”, International Journal of Thermophysics 2005; 26(1): 31-46, is as follows:

P/ρₘ=Pₘ=A_{ε}+A_{μ}*/T+B_{ε}·ρₘ+C·ρₘ^{D}

In this case the electric polarisation P can be expressed as

P=Pₘρₘ=(εᵣ−1)/(εᵣ+2).

Furthermore A_{μ}*=N_{A}μ²/9ε₀k_{B}=A_{μ}T should clarify the temperature dependence of the coefficient A_{μ}. The virial coefficients A_{ε}, B_{ε} and C as empirical parameter (in contrast to the equations 8 and 10) were made temperature dependent as follows:

$$\begin{matrix}{A}_{ɛ} = {a}_{0} + {a}_{1} ( \frac{T}{{T}_{0}} - 1 ) & ( 12 a ) \\ {B}_{ɛ} = {b}_{0} + {b}_{1} ( \frac{{T}_{0}}{T} - 1 ) & ( 12 b ) \\ C = {c}_{0} + {c}_{1} ( \frac{{T}_{0}}{T} - 1 ) & ( 12 c )\end{matrix}$$

Measurements were performed with (1) nitrogen and (2) methane N55 (research methane with high purity) at pressures between 0.1 MPa and 30/35 MPa.

The methane (pressurised gas cylinder with 20 MPa pressure and 50 l volume) had the following composition:
CH₄ ≤99.9995 Vol.-%
N₂ ≤2 ppmv
O₂ ≤0.5 ppmv
H₂O ≤2 ppmv
CO₂ ≤0.1 ppmv
Other hydrocarbons ≤0.15 ppmv

The required parameters for the correlation of the molar polarisation Pₘ with equation 11 are given by Harvey A H, Lemmon E W, “Method for Estimating the Dielectric Constant of Natural Gas Mixtures”, International Journal of Thermophysics 2005; 26(1): 31-46. Table 1 is an extract from this table which includes the data for nitrogen and methane.

TABLE 1
Parameters for the correlation of molar polarisation Pₘ with equation [symbol] in accordance with Harvey
and Lemmon [27]

| | a₀ | a₁ | A_{μ}* | b₀ | b₁ | c₀ | c₁ | |
|---|---|---|---|---|---|---|---|---|
| Fluid | cm³ mol⁻¹ | cm³ mol⁻¹ | cm³ mol⁻¹ K | cm⁶mol⁻² | cm⁶mol⁻² | cm⁶mol⁻² | cm^{3(D+1)} mol^{-(D+1)} | D |
| N₂ | 4.3872 | 0.00226 | 0 | 2.2060 | 1.1350 | −169.00 | −35.83 | 2.1 |
| CH₄ | 6.5443 | 0.01330 | 0 | 8.4578 | 3.7196 | −352.97 | −100.65 | 2.0 |

A summary of the measurement results for the measurements with nitrogen can be found in Table 2 which shows a comparison of measured values εₘₑₐₛ with theoretical values εₜₕₑₒ for pressures in the range between 0.01 MPa to 29.96 MPa and temperatures between 12.8° C. to 20.1° C. The data for the required molar density ρₘ were obtained from P. J. Linstrom and W. G. Mallard (editors): NIST Chemistry WebBook, NIST Standard Reference Database Number 69, National Institute of Standards and Technology (NIST), 2005 (http://webbook.nist.gov/chemistry) considering the respective pressure and temperature.

TABLE 2
Comparison of the theoretical dielectric constant ϵ_{theo.} according to Harvey and Lemmon [symbol] with
the measured dielectric constant ϵ_{meas.} for nitrogen.

| Temp. | Pressure | ρₘ | | | | | | ϵ_{theo.} | Microfactor | ϵ_{meas.} |
|---|---|---|---|---|---|---|---|---|---|---|
| ° C. | MPa | mol m⁻³ | A_{ϵ} | A_{μ}* | B_{ϵ} | C | Pₘ | — | — | — |
| 12.8 | 0.01 | 4.2062 | 4.38731 | 0 | 2.15520 | −167.39620 | 0.000018 | 1.00006 | 1.0000 | 1.00000 |
| 13.8 | 2.53 | 1066.8417 | 4.38731 | 0 | 2.15142 | −167.27692 | 0.064683 | 1.01411 | 0.9943 | 1.01150 |
| 13.7 | 5.12 | 2165.7068 | 4.38731 | 0 | 2.15179 | −167.28881 | 0.009510 | 1.02880 | 0.9878 | 1.02485 |
| 15.5 | 10.30 | 4298.6210 | 4.38733 | 0 | 2.14505 | −167.07606 | 0.018886 | 1.05775 | 0.9749 | 1.05216 |
| 17.0 | 15.19 | 6178.7653 | 4.38734 | 0 | 2.13950 | −166.90078 | 0.027151 | 1.08373 | 0.9638 | 1.07653 |
| 17.8 | 20.09 | 7903.7870 | 4.38735 | 0 | 2.13656 | −166.80804 | 0.034728 | 1.10793 | 0.9545 | 1.09761 |
| 20.1 | 25.00 | 9385.3959 | 4.38737 | 0 | 2.12821 | −166.54422 | 0.041227 | −1.12900 | 0.9459 | 1.11766 |
| 18.6 | 25.47 | 9582.3729 | 4.38735 | 0 | 2.13364 | −166.71580 | 0.042090 | 1.13182 | 0.9443 | 1.12145 |
| 20.1 | 27.58 | 10132.9289 | 4.38737 | 0 | 2.12821 | −166.54422 | 0.044502 | 1.13972 | 0.9412 | 1.12885 |
| 20.1 | 29.96 | 10781.5066 | 4.38737 | 0 | 2.12821 | −166.54422 | 0.047341 | 1.14908 | 0.9382 | 1.13608 |

A compilation of the measurement results regarding the investigations with methane using the same method can be found in Table 3 for pressures in the range between 0.07 MPa to 35.04 MPa and temperatures between 10.5° C. to 20.8° C.

TABLE 3
Comparison of the theoretical dielectric constant ϵ_{theo.} according to Harvey and Lemmon [symbol] with
the measured dielectric constant ϵ_{meas.} for methane.

| Temp. | Pressure | ρₘ | | | | | | ϵ_{theo.} | Microfactor | ϵ_{meas.} |
|---|---|---|---|---|---|---|---|---|---|---|
| ° C. | MPa | mol m⁻³ | A_{ϵ} | A_{μ}* | B_{ϵ} | C | Pₘ | — | — | — |
| 10.5 | 0.07 | 29.7240 | 6.54481 | 0 | 8.32011 | −349.24432 | 0.000195 | 1.00058 | 1.0000 | 1.00000 |
| 13.3 | 2.64 | 1169.3912 | 6.54495 | 0 | 8.28510 | −348.29694 | 0.007664 | 1.02317 | 0.9916 | 1.01701 |
| 15.4 | 5.32 | 2462.8558 | 6.54505 | 0 | 8.25929 | −347.59847 | 0.016164 | 1.04929 | 0.9804 | 1.04038 |
| 17.9 | 10.00 | 4946.7632 | 6.54517 | 0 | 8.22905 | −346.78009 | 0.032537 | 1.10089 | 0.9580 | 1.08960 |
| 19.4 | 15.01 | 7687.4424 | 6.54524 | 0 | 8.21115 | −346.29578 | 0.050644 | 1.16004 | 0.9333 | 1.14804 |
| 20.1 | 20.14 | 10168.1649 | 6.54528 | 0 | 8.20286 | −346.07146 | 0.067038 | 1.21556 | 0.9131 | 1.19940 |
| 20.4 | 25.00 | 12039.8379 | 6.54529 | 0 | 8.19932 | −345.97565 | 0.079389 | 1.25871 | 0.8993 | 1.23649 |
| 20.7 | 29.99 | 1353.5633 | 6.54531 | 0 | 8.19579 | −345.88004 | 0.089225 | 1.29390 | 0.8861 | 1.27360 |
| 20.8 | 35.04 | 14741.9288 | 6.54531 | 0 | 8.19461 | −345.84821 | 0.097163 | 1.32286 | 0.8758 | 1.30374 |

The actual temperatures were taken into account for the calculation of the theoretical value of the relative permittivity (dielectric constant) in all cases.

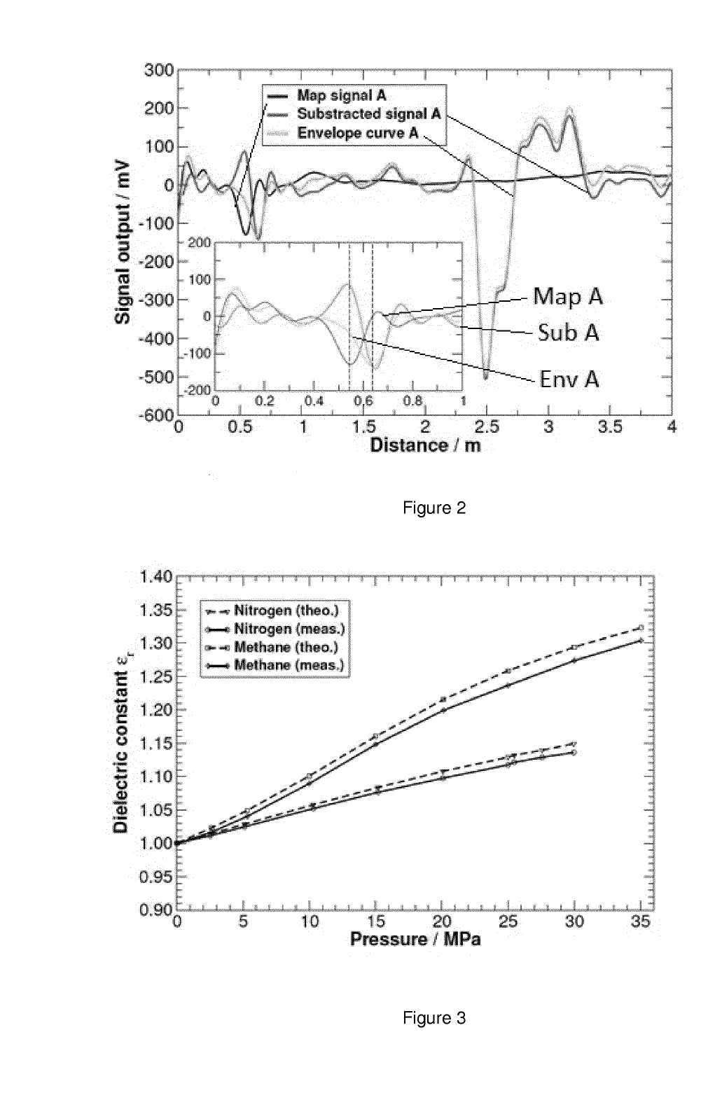

The determination of the microfactor and hence the dielectric constant was performed using the signal data (envelope curves) from the GWR system (see FIG. 2 as example for the measurement of methane at 35.04 MPa at a temperature of 20.8° C.). As can be seen the “peak” obtained from the reference position (reference rod) was shifted from 545 mm (map signal A) to 638 mm (envelope curve A). Due to the construction of the system of FIG. 1, the dielectric of the gas also affects the signal above the zero reference point of the flange face 16. The correction factor (microfactor) can be calculated according equation 5 with consideration of the additional distance d_{F} to account for the length of the section 24.

$$microfactor = \frac{Physical reference distance + {d}_{F}}{Apparent reference distance + {d}_{F}} \qquad ( 13 )$$

The value d_{F} mainly depends on the flange size. For the system of FIG. 1 used for these tests, the additional distance has a value of d=96 mm. The result is a microfactor of 0.8733 or a corresponding value for the dielectric constant εᵣ of 1.31122 (see equation 6). A comparison of the values is presented in FIG. 3 including measurement data for nitrogen and methane. As can be seen, the measured values have the same characteristic curve as the theoretical values but are below (negative offset) the theoretical curve in both cases.

The bias or systematic error can have many causes. In the case of the system used for the experimental results quoted, the reference rod 22 (500 mm) was installed at a distance of 45 mm from the inner side of the top flange. This results in a physical reference rod distance of 545 mm. Due to the design of the system, the coupling of the GWR signal is already at the fixed distance d_{F}=96 mm above the flange. This distance, as recommended value provided by the manufacture of the level meter, is in addition to the design of the level meter mainly based on the thickness of the connection flange 16. As can be seen in the Tables 2 and 3 there is already a deviation between the theoretical and measured dielectric constants at (approximately) atmospheric pressure. These deviations are apparently related to an incorrect d_{F} value. The determination of a new d_{F} from the measured values by using a least squares method results in better results. With the calculated value the curve of theoretical dielectric constant and the curve of measured dielectric constant agree within an expanded uncertainty U(k=2) of 0.5%. Another source of uncertainty is the method itself. The measurement uncertainty can be reduced by using a greater Physical reference distance (distance to the end of the reference rod 22). Any measurement error present in measurement of the reference rod 22 will lead to a measurement error in the measured distance which has to be taken into account. Consequently, the greater the distance to the reference section the smaller the potential error.

GWR devices have been proposed for a number of process monitoring uses. In certain cases, determination of relative permittivity has also been proposed to improve level measurement results. Examples can be found in WO 2016/011531, WO 2004/06663, WO 00/43806, WO 2016/011530, WO 00/437739, GB 2358535, WO 01/18533, US 2009/0303106, and US 2005/0230619.

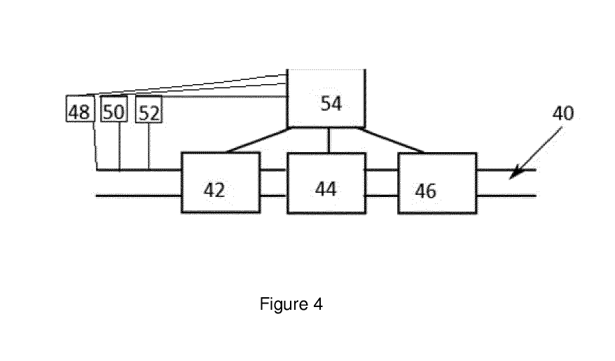

FIG. 4 shows a system for measuring the energy content of a gas according to one embodiment of the invention. The system comprises a pipeline 40 through which a gas such as natural gas (NG) flows. A series of sensor systems are mounted on the pipeline 40 to measure the properties of the flowing gas. A first sensor system 42 measures the speed of sound in the gas. This can comprise an ultrasonic flow meter or other speed of sound sensor. The second sensor system 44 comprises a GWR or non-GWR (non-contacting radar) sensor for determining the relative permittivity of the gas. While the GWR systems discussed above comprise level sensors (i.e. they measure the reflections from the liquid surface), it will be clear that as the relative permittivity can be obtained from the reference measurement, a similar sensor could be used in a gas-only environment (i.e. in which there is no liquid surface present). A third sensor system 46 provides the mole fraction of carbon dioxide. This can comprise a CO₂ sensor, a non-dispersive infrared (NDIR) sensor, a photoacoustic CO₂ sensor, a tuneable laser diode absorption spectroscopy (TLDAS) device, or an on-line infrared gas analyser. Further sensors 48, 50, 52 provide temperature, pressure, and flow rate data. The outputs of the various sensor systems are provided as inputs to an electronics module 54 including data processing capability configures as a correlator to correlate the various measurement in the manner described above to obtain the energy content and energy of the gas. In certain cases, the first sensor system can provide flow rate data, in which case a separate sensor 52 can be omitted.—In certain cases, sensor 52 provides speed-of-sound data, in which case a separate sensor 42 can be omitted.

One variant of this system is in connection with the measurement of the energy content of Liquefied Natural Gas (LNG). LNG has, in contrast to natural gas, a much lower share of nitrogen and carbon dioxide since nitrogen and carbon dioxide have to be removed before the final liquefaction process. Due to the risk of “auto-stratification” the nitrogen content of LNG is kept under 1%. The removal of carbon dioxide from LNG is essential in order to prevent “freeze-out” during the liquefaction process since carbon dioxide would freeze at cryogenic temperatures and could clog the liquefaction equipment such as the heat exchangers. Consequently, the carbon dioxide content is kept to no more than 50 ppm. This means the share of carbon dioxide is therefore negligible for LNG. For the same reason, heavier hydrocarbons are also stripped out so that only methane and some light hydrocarbons remain. Therefore, LNG can be seen in good approximation only as a mixture of light hydrocarbons (C1-C4) and nitrogen, with methane as main share.

Level measurement is a known technology with regard to LNG, especially related to storage tank metering and ship tank level metering where the loaded and unloaded volume of LNG is measured static by means of level differences. By using an appropriate GWR or non-GWR (non-contacting radar) level meter the dielectric constant of the LNG can also be obtained. Because the carbon dioxide content of LNG is so low, its measurement is unnecessary, the measurement of the speed of sound w and the relative permittivity & being sufficient for all relevant gas properties to be determined by means of correlation methods. For LNG applications, specifically designed ultrasonic flow meters are available for measuring the speed of sound on-line (and real time) under process conditions. The GWR or non-GWR (non-contacting radar) method is particularly suitable for the on-line (and real-time) measurement of relative permittivity & of LNG. The method of the invention allows the energy content of LNG to be extracted from existing flow and level measurements made during the transportation of LNG, such as during loading and/or offloading of carrier vessels. In addition, the use of measurements from a GWR level sensor can give two separate readings that are affected by εᵣ: the echo from the reference rod, and the echo from the liquid surface. While GWR or non-GWR (non-contacting radar) level measurement systems used for LNG typically only considers the microfactor, so as to correct the level measurement, the method of the invention provides a technique in which this “extra” measurement can also be used to obtain εᵣ for use in the correlation to determine energy content.

It will be appreciated that various changes can be made to the embodiments described above while remaining within the scope of the claims.

1. A method of measuring the energy content of a gas, comprising:
measuring the speed of sound in the gas;
measuring the relative permittivity of the gas using a guided wave radar or non-guided wave (non-contacting) radar sensor; and
correlating the measured speed of sound and relative permittivity to derive the energy content of the gas.

2. A method as claimed in claim 1, further comprising measuring pressure and temperature parameters of the gas, and using the parameters to derive the energy content of the gas.

3. A method as claimed in claim 1 or 2, wherein the gas is flowing in a conduit, the measurements being made on the flowing gas.

4. A method as claimed in claim 3, further comprising measuring flow rate parameters, and using the flow rate parameters, together with the other measurements, to derive the energy content of the gas.

5. A method as claimed in claim 1 or 2, wherein the gas comprises liquefied gas in a container, the measurement of relative permittivity being derived from a level measurement using the guided wave radar sensor.

6. A method as claimed in claim 5, wherein the speed of sound measurement is made as part of a flow measurement during loading of liquefied gas into the container, or during unloading of liquefied gas from the container.

7. A method as claimed in any preceding claim, wherein measuring the relative permittivity of the gas comprises a time domain reflectometry measurement of a microwave pulse in a wave guide.

8. A method as claimed in any preceding claim, further comprising measuring the carbon dioxide content of the gas, and correlating the measured speed of sound, relative permittivity, and carbon dioxide content to derive the energy content of the gas.

9. A system for measuring the energy content of a gas, comprising:
a first sensor for measuring the speed of sound in the gas;
a second sensor for measuring the relative permittivity of the gas using a guided wave radar sensor; and
a correlator configured to correlate the measured speed of sound and relative permittivity to derive the energy content of the gas.

10. A system as claimed in claim 9, further comprising sensors for measuring pressure and temperature parameters of the gas, and wherein the correlator is configured to use the parameters to derive the energy content of the gas.

11. A system as claimed in claim 9 or 10, further comprising a conduit housing the sensors and through which the gas flows.

12. A system as claimed in claim 11, further comprising further sensors for measuring volumetric flow parameters, wherein the correlator is configured to use the volumetric flow parameters and the other measurements to derive the energy content of the gas.

13. A system as claimed in claim 9 or 10, wherein the gas comprises liquefied gas in a container, the second sensor comprising a guided wave radar level measurement sensor.

14. A system as claimed in claim 13, wherein the first sensor comprises a flow measurement sensor for use during loading of liquefied gas into the container, or during unloading of liquefied gas from the container.

15. A system as claimed in any of claims 9-14, wherein the second sensor comprises a microwave source and a waveguide of predetermined configuration, the second sensor being configured to provide a time domain reflectometry measurement of a microwave pulse reflected by a predetermined feature of the waveguide configuration for determining the relative permittivity of the gas.

16. A system as claimed in claim 15, wherein the predetermined feature is the end of a reference rod mounted on the waveguide.

17. A system as claimed in any of claims 9-16, further comprising a third sensor for measuring the carbon dioxide content of the gas, and wherein the correlator is configured to use the measured speed of sound, relative permittivity, and carbon dioxide content to derive the energy content of the gas.

18. A system as claimed in claim 17, wherein the third sensor comprises an infrared sensor.

19. A system as claimed in any of claims 9-18, wherein the first sensor comprises an ultrasonic flow meter.

20. A system as claimed in any of claims 9-19, further comprising a processor configured to receive the output of the correlator as an input, and to output values of superior calorific value, Wobbe index and normal density of the gas.
